(19) United States
(12) Patent Application Publication
(10) Pub. No.: US 2022/0336063 A1
(43) Pub. Date: Oct. 20, 2022

(54) MEDICAL INFORMATION PROCESSING APPARATUS

(71) Applicant: CANON MEDICAL SYSTEMS CORPORATION, Otawara-shi (JP)

(72) Inventors: Kazumasa NORO, Shioya-gun (JP); Yusuke KANO, Nasushiobara (JP); Sho SASAKI, Utsunomiya (JP); Yudai YAMAZAKI, Nasushiobara (JP); Kosuke ARITA, Otawara-shi (JP)

(73) Assignee: CANON MEDICAL SYSTEMS CORPORATION, Otawara-shi (JP)

(21) Appl. No.: 17/658,268

(22) Filed: Apr. 7, 2022

(30) Foreign Application Priority Data
Apr. 16, 2021 (JP) 2021-069631

(51) Int. Cl. G16H 10/60

(52) U.S. Cl. CPC G16H 10/60

(57) ABSTRACT

A medical information processing apparatus comprises processing circuitry. The processing circuitry extracts a character string having clinical meaning from first medical data indicating text data included in medical information, as a topic indicating a reference viewpoint of the medical information. The processing circuitry selects one of the topics from the extracted topics according to a user input. The processing circuitry, among second medical data that includes one or more pieces of data representing a condition of a subject to be examined and that is included in the medical information, performs control of displaying the second medical data related to one of the selected topics on a display device.

### CROSS-REFERENCE TO RELATED APPLICATIONS

This application is based upon and claims the benefit of priority from Japanese Patent Application No. 2021-069631, filed on Apr. 16, 2021; the entire contents of which are incorporated herein by reference.

### FIELD

Embodiments described herein relate generally to a medical information processing apparatus.

### BACKGROUND

In recent years, there are various types of medical information such as sample test data, medical image data, and electronic medical record data used in the medical field. Doctors will diagnose, treat a subject (for example, a patient), or perform any other actions by comprehensively looking at such information. Thus, for example, to improve the efficiency of diagnosis, a technology has been developed to display various types of medical data necessary for a doctor to make a diagnosis, on a single screen with information to support the diagnosis.

However, if there is a change in the reference viewpoint of the medical information (for example, if a doctor wishes to “examine treatment plans after the diagnosis is made” or the like), the doctor needs to collect necessary medical information again, according to the reference viewpoint of the medical information. In this manner, reference on the medical information may be made inefficiently, and important medical information may be overlooked.

### BRIEF DESCRIPTION OF THE DRAWINGS

FIG. 1 is a block diagram illustrating an example of a configuration of a medical information processing apparatus according to a first embodiment;

FIG. 2 is an image illustrating an example of an extraction process of a topic according to the first embodiment;

FIG. 3 is a diagram illustrating an example of a related topic display screen according to the first embodiment;

FIG. 4 is a diagram illustrating an example of a process for determining medical data related to the selected topic according to the first embodiment;

FIG. 5 is an image illustrating an example of medical data classification and medical data display according to the first embodiment;

FIG. 6 is a diagram illustrating an example of a data display screen according to the first embodiment;

FIG. 7 is a diagram illustrating an example of the data display screen according to the first embodiment;

FIG. 8 is an image illustrating an example of a topic search process according to the first embodiment;

FIG. 9 is a flowchart illustrating an example of a process executed by the medical information processing apparatus according to the first embodiment;

FIG. 10 is a block diagram illustrating an example of a configuration of a medical information processing apparatus according to a second embodiment;

FIG. 11 is an image illustrating an example of an aggregation process of equivalent topics according to the second embodiment;

FIG. 12 is a flowchart illustrating an example of a process executed by the medical information processing apparatus according to the second embodiment;

FIG. 13 is a block diagram illustrating an example of a configuration of a medical information processing apparatus according to a third embodiment;

FIG. 14 is an image illustrating an example of an aggregation process of related topics according to the third embodiment;

FIG. 15 is an image illustrating an example of a generation process of related topic groups according to the third embodiment;

FIG. 16 is an image illustrating an example of a related topic display screen according to the third embodiment;

FIG. 17 is an image illustrating an example of the related topic display screen according to the third embodiment; and

FIG. 18 is a flowchart illustrating an example of a process executed by the medical information processing apparatus according to the third embodiment.

### DETAILED DESCRIPTION

A medical information processing apparatus comprises processing circuitry. The processing circuitry extracts a character string having clinical meaning from first medical data indicating text data included in medical information, as a topic indicating a reference viewpoint of the medical information. The processing circuitry selects one of the topics from the extracted topics according to a user input. The processing circuitry, among second medical data that includes one or more pieces of data representing a condition of a subject to be examined and that is included in the medical information, performs control of displaying the second medical data related to one of the selected topics on a display device.

Hereinafter, embodiments of a medical information processing apparatus will be described in detail with reference to the accompanying drawings.

### First Embodiment

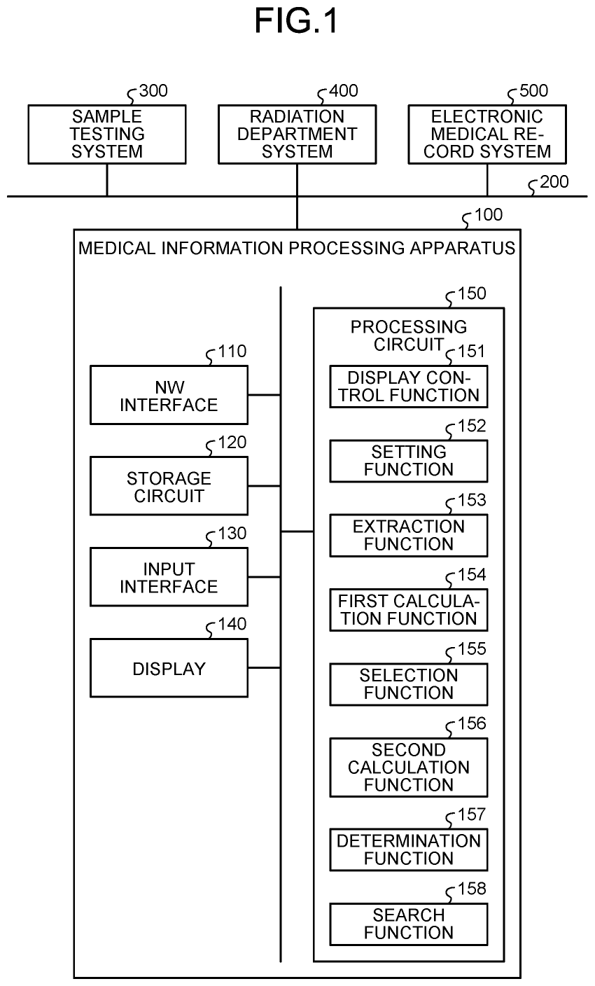

FIG. 1 is a diagram illustrating an example of a configuration of a medical information processing apparatus according to a first embodiment. For example, as illustrated in FIG. 1, a medical information processing apparatus 100 according to the present embodiment is communicatively connected to a sample testing system 300, a radiation department system 400, an electronic medical record system 500, and the like via a network 200. For example, the medical information processing apparatus 100 and the systems are installed in a hospital and the like, and are connected to each other by the network 200 such as an in-hospital LAN.

The sample testing system 300 generates medical data on a sample test performed on a subject, and stores the generated medical data in a memory in the system. Then, corresponding to a request from the medical information processing apparatus 100, the sample testing system 300 transmits the medical data stored in the memory to the medical information processing apparatus 100.

The radiation department system 400 generates medical data on vital and imaging examinations performed on the subject, and stores the generated medical data in the memory in the system. For example, the radiation department system 400 includes picture archiving and communication systems (PACS).

Moreover, the imaging examination includes an examination using a computed tomography (CT) image, which is taken by an X-ray CT device, an examination using a magnetic resonance (MR) image, which is taken by a magnetic resonance imaging (MRI) device, an examination using an ultrasonic image, which is taken by an ultrasonic diagnostic device, and an examination using an X-ray image, which is taken by an X-ray diagnostic device.

Corresponding to a request from the medical information processing apparatus 100, the radiation department system 400 transmits the medical data stored in the memory to the medical information processing apparatus 100.

The electronic medical record system 500 generates medical data on prescription and nursing records of the subject, and stores the generated medical data in the memory in the system. Then, corresponding to a request from the medical information processing apparatus 100, the electronic medical record system 500 transmits the medical data stored in the memory to the medical information processing apparatus 100.

The medical information processing apparatus 100 acquires various types of medical data from the sample testing system 300, the radiation department system 400, and the electronic medical record system 500 via the network 200, and performs various types of information processing using the acquired medical data. For example, the medical information processing apparatus 100 is implemented by a computer device such as a workstation, a personal computer, or a tablet terminal.

Specifically, the medical information processing apparatus 100 includes a network (NW) interface 110, a memory 120, an input interface circuit 130, a display device 140, and a processing circuit 150.

The NW interface circuit 110 is connected to the processing circuit 150, and controls the transmission and communication of various types of data performed between the medical information processing apparatus 100 and each system. Specifically, the NW interface circuit 110 receives medical data from each system, and outputs the received medical data to the processing circuit 150. For example, the NW interface circuit 110 is implemented by a network card, a network adapter, a network interface controller (NIC), and the like.

The memory 120 is connected to the processing circuit 150, and stores various types of data. Specifically, the memory 120 stores the medical data received from each system. For example, the memory 120 is implemented by a semiconductor memory device such as a random access memory (RAM) and flash memory, a hard disk, an optical disc, and the like.

The input interface circuit 130 is connected to the processing circuit 150, and receives various instructions and input operations of various types of information from an operator. Specifically, the input interface circuit 130 converts an input operation received from the operator into an electrical signal, and outputs the electrical signal to the processing circuit 150.

For example, the input interface circuit 130 is implemented by a trackball, a switch button, a mouse, a keyboard, a touch pad with which input operations are performed by touching an operation surface, a touch screen in which a display screen and a touch pad are integrated, a non-contact input circuit using an optical sensor, a voice input circuit, and the like. In the present specification, the input interface circuit 130 is not limited to one having physical operation parts such as a mouse and a keyboard.

For example, an electrical signal processing circuit that receives an electrical signal corresponding to an input operation from an external input device, which is provided separately from the device, and that outputs the electrical signal to a control circuit, is also an example of the input interface circuit 130.

The display device 140 is connected to the processing circuit 150 and displays various types of information and images. Specifically, the display device 140 converts data on various types of information and images sent from the processing circuit 150 into electrical signals for display, and outputs the electrical signals. For example, the display device 140 is implemented by a liquid crystal monitor, a cathode ray tube (CRT) monitor, a touch panel, and the like. The display device 140 is an example of a display device.

The processing circuit 150 controls the components of the medical information processing apparatus 100, corresponding to the input operation received from the operator via the input interface circuit 130. Specifically, the processing circuit 150 stores the medical data output from the NW interface circuit 110 into the memory 120. Moreover, the processing circuit 150 reads the medical data from the memory 120, and displays the read medical data on the display device 140. For example, the processing circuit 150 is implemented by a processor.

The overall configuration of the medical information processing apparatus 100 according to the present embodiment has been described. Under such a configuration, the medical information processing apparatus 100 according to the present embodiment is configured so that the operator such as a doctor can efficiently refer to necessary medical information.

Specifically, in the present embodiment, the memory 120 stores integrated medical database (DB) including various types of medical data acquired from the sample testing system 300, the radiation department system 400, and the electronic medical record system 500. In this example, the medical data stored in the integrated medical DB includes information such as numerical values (measured values), images, and medical records, and information indicating the recorded date and time of the numerical values (measured values), the images, and the medical records.

For example, the integrated medical DB includes sample test data, vitals data, medical image data, prescription data, and nursing record data. The sample test data is medical data on the sample test obtained from the sample testing system 300. The vitals data is medical data on vitals obtained from the radiation department system 400. The medical image data is medical data on imaging examination obtained from the radiation department system 400.

The prescription data is medical data on prescriptions obtained from the electronic medical record system 500. The nursing record data is medical data on nursing record obtained from the electronic medical record system 500.

The medical data stored in the integrated medical DB may be data obtained from each of the sample testing system 300, the radiation department system 400, and the electronic medical record system 500; may be integrated data obtained from the systems; or may be information created for the purpose of secondary use.

Moreover, the memory 120 stores display information that indicates the screen size of the display device 140 and the like. Furthermore, the memory 120 stores classification information that indicates the classification of sample test data, vitals data, medical image data, prescription data, nursing record data, and the like. Still furthermore, the memory 120 stores item number information that indicates the maximum number of displayable medical data (item) for each screen size.

Then, in the present embodiment, the processing circuit 150 has a display control function 151, a setting function 152, an extraction function 153, a first calculation function 154, a selection function 155, a second calculation function 156, a determination function 157, and a search function 158.

The display control function 151 performs control of displaying various types of information on the display device 140. The details of the display process will be described with the following description of each function.

The setting function 152 sets an extraction condition indicating the condition for extracting a character string having clinical meaning, corresponding to the reference purpose of the medical information. The setting function 152 sets a character string input by a user as an extraction condition.

For example, if a patient to be examined is suffering from “Tetralogy of Fallot”, and if a user (doctor) wishes to refer to information on the “Tetralogy of Fallot”, the user inputs “Tetralogy of Fallot” as an extraction condition. In this case, the setting function 152 sets the “Tetralogy of Fallot” as an extraction condition.

In this example, the extraction condition is the disease name. However, the extraction condition may also be a patient ID for identifying a patient, a doctor ID for identifying a doctor in charge of medical treatment, the clinical department name indicating the clinical department, or the like. Moreover, a combination of the patient ID, the doctor ID, the clinical department name, or the like may be used as the extraction condition. Furthermore, the setting function 152 may specify the doctor in charge, the clinical department, or the like from a user ID for identifying a user of the medical information processing apparatus 100, and automatically set the doctor in charge, the clinical department, or the like as an extraction condition.

The extraction function 153 extracts a character string having clinical meaning, from first medical data indicating text data included in medical information, which indicates various types of data used for medical treatment, as a topic indicating the reference viewpoint of the medical information.

For example, the first medical data includes text data included in the medical data (an example of medical information) such as sample test data, vitals data, prescription data, and nursing record data. Moreover, the other first medical data includes metadata (such as a Digital Imaging and Communications in Medicine (DICOM) tag) that indicates the attribute of medical image data (an example of medical information).

Specifically, the extraction function 153 extracts a character string having clinical meaning from the first medical data as a topic, according to the extraction condition indicating the condition for extracting a character string having clinical meaning, corresponding to the reference purpose of the medical information.

More specifically, on the basis of the extraction condition set by the setting function 152, the extraction function 153 extracts a character string having clinical meaning from the first medical data, by a known natural language processing technique (such as unique expression extraction) using language resources such as dictionaries, corpora, and ontologies.

When the extraction condition is set as a character string indicating the disease name or the like, the extraction function 153 also extracts a character string related to equivalent terms, synonyms, and abbreviations of the character string indicating the disease name or the like as a topic.

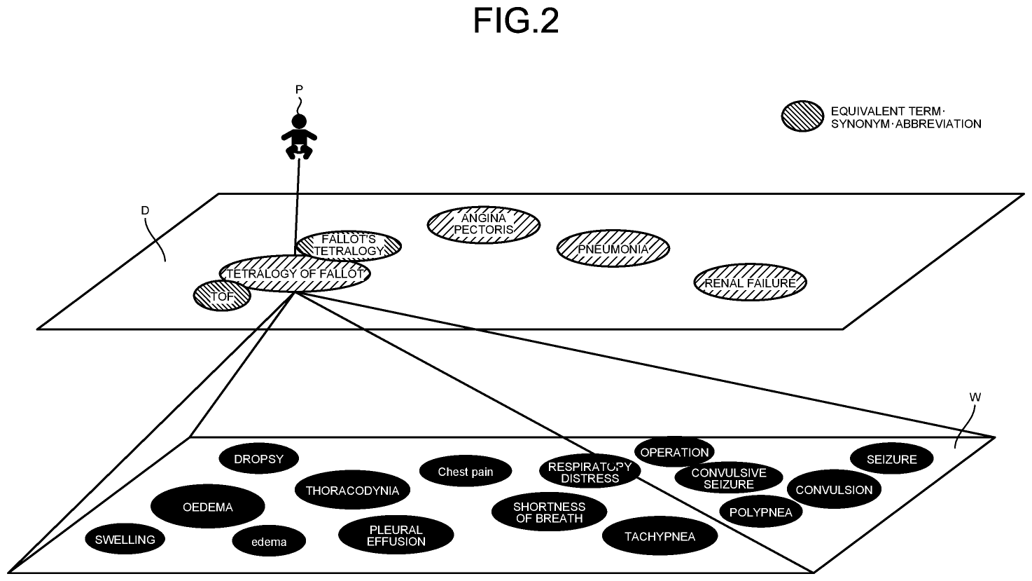

Hereinafter, extraction of a topic will be described with reference to FIG. 2. FIG. 2 is an image illustrating an example of an extraction process of a topic.

In this example, “Tetralogy of Fallot”, is one of one or more of diseases D related to a patient P to be examined, and a doctor inputs the “Tetralogy of Fallot” as an extraction condition of a topic. In this case, the setting function 152 first sets the “Tetralogy of Fallot” as an extraction condition C.

The extraction function 153 refers to the integrated medical DB, and from the text data included in the medical data such as sample test data, extracts a character string W having clinical meaning such as oedema, thoracodynia, and shortness of breath related to “Tetralogy of Fallot”, “Fallot's tetralogy”, which is a term equivalent to the “Tetralogy of Fallot”, and “TOF”, which is an abbreviation of “Tetralogy of Fallot”, as a topic, using a known natural language processing technique.

Returning to FIG. 1, the description will continue. The first calculation function 154 calculates the level of importance indicating a degree of importance of the topic. Specifically, the first calculation function 154 calculates the level of importance of each topic extracted by the extraction function 153, on the basis of appearance probability and reference probability of the topic.

Hereinafter, a calculation process of the level of importance will be described. In this example, the level of importance of a topic “oedema” will be calculated. In this case, the first calculation function 154 first calculates the appearance probability of “oedema”. For example, the first calculation function 154 calculates the appearance probability of “oedema”, from the ratio of the total number (total sum) of “oedema” extracted by the extraction function 153 with respect to the total number (total sum) of topics extracted by the extraction function 153.

In terms of probability of the topic “oedema” appearing on the patient to be examined, the first calculation function 154 may also calculate the appearance probability of “oedema”, from the ratio of the total number of “oedema” extracted from the first medical data of the patient to be examined by the extraction function 153, with respect to the total number of “oedema” extracted by the extraction function 153.

Moreover, in terms of probability of the topic “oedema” appearing during a certain period of time, the first calculation function 154 may also calculate the appearance probability of “oedema”, from the ratio of the total number of “oedema” extracted during a certain period of time (for example, the most recent month) by the extraction function 153, with respect to the total number of “oedema” extracted by the extraction function 153. Furthermore, depending on a situation, different calculation methods described above may be used, or multiple calculation methods may be combined.

Next, the first calculation function 154 calculates the reference probability of “oedema”. For example, the first calculation function 154 calculates the reference probability of “oedema”, from the ratio of the total number (total sum) of references made to “oedema” by the user, with respect to the total number (total sum) of references made to the topic by the user.

In terms of probability of the topic “oedema” being referred to by the user in the medical treatment of the patient to be examined, the first calculation function 154 may also calculate the reference probability of “oedema”, from the ratio of the total number of references made to “oedema” in the medical treatment of the patient to be examined, with respect to the total number of references made to “oedema” by the user.

Moreover, in terms of probability of the topic “oedema” being referred to during a certain period of time, the first calculation function 154 may also calculate the reference probability of “oedema”, from the ratio of the total number of references made to “oedema” during a certain period of time (for example, the most recent month), with respect to the total number of references made to “oedema” by the user. Furthermore, depending on a situation, different calculation methods described above may be used, or multiple calculation methods may be combined.

Then, the first calculation function 154 calculates the level of importance of “oedema” on the basis of the sum of the appearance probability of “oedema” and the reference probability of “oedema”. The appearance probability and the reference probability may be weighted individually, depending on which one of the appearance probability and the reference probability is considered more important.

Then, the display control function 151 performs control of rearranging the topics and displaying the topics on the display device 140, according to the level of importance of the topics calculated by the first calculation function 154.

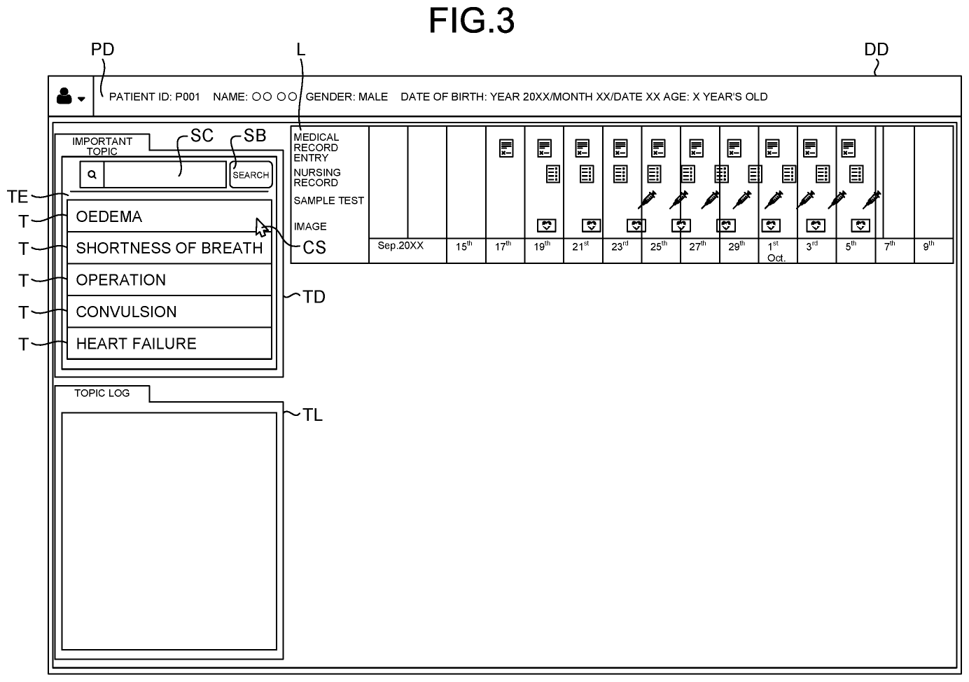

Hereinafter, a display process of topics will be described with reference to FIG. 3. FIG. 3 is a diagram illustrating an example of a topic display screen.

When the extraction function 153 extracts topics, as illustrated in FIG. 3, the display control function 151 displays The patient data field PD, the topic display panel TD, the topic log panel TL, and the medical data list L on the data display screen DD displayed on the display device 140.

The patient data field PD is a display field indicating the basic information of the patient to be examined, such as the name of the patient to be examined. In this example, the display control function 151 displays the basic information of “name: XXXX” identified by the “patient ID: P001” in the patient data field PD.

The topic display panel TD is a display field for the item related to the topic. The display control function 151 displays the search box SC, the search button SB, the topic display area TE, and the cursor CS in the topic display panel TD. The search box SC, the search button SB, and the cursor CS will be described below.

In this example, the display control function 151 displays “oedema”, “shortness of breath”, “operation”, “convulsion”, and “heart failure” in the topic display area TE as the topics T, in the descending order of the level of importance calculated by the first calculation function 154.

The topic log panel TL is a display field for displaying a topic history indicating the topic that has been referred to in the past (past topic). The topic log panel TL will be described below. The medical data list L is a list of medical data of the patient to be examined (in the example of FIG. 3, the patient identified by “patient ID: P001”). The user can display any medical data on the data display screen DD, by selecting and clicking an icon in the medical data list L.

Returning to FIG. 1, the description will continue. The selection function 155 selects one topic from the topics extracted by the extraction function 153 according to the user input.

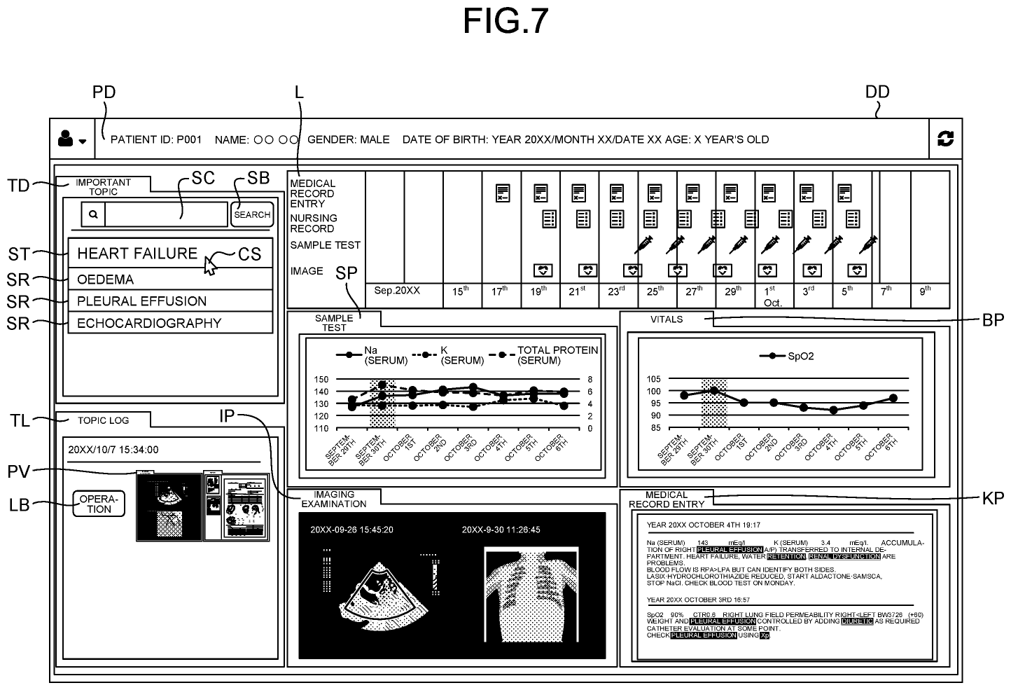

Specifically, the selection function 155 selects the topic T displayed in the topic display area TE according to the user operation. In the example of FIG. 7, if the user places the cursor CS on “operation”, which is one of the topics T, and left-clicks the mouse, the selection function 155 selects “topic: operation”. For example, if the user left clicks the mouse on a past topic displayed on the topic log panel TL, the selection function 155 selects the past topic.

The second calculation function 156 calculates the degree of association indicating a degree of relation between the topics. Specifically, the second calculation function 156 calculates the degree of association between the topic selected by the selection function 155 and another topic.

More specifically, the second calculation function 156 analyzes the relevance between the topics extracted by the extraction function 153, using a known analysis method such as association analysis, co-occurrence network, causal inference (inverse probability of treatment weighting (IPTW) method, doubly robust (DR) method), causal search (Linear Non-Gaussian Acyclic Model (LinGAM), Bayesian network), knowledge graph, principal component analysis (PCA), topic model (latent dirichlet allocation (LDA)), latent semantic analysis (LSA), and distributed representation (Word2Vec), and calculates the degree of association between the topics on the basis of the analysis result.

When the selection function 155 selects one topic, the display control function 151 displays topics (hereinafter, also referred to as related topics) in which the degree of association with the topic exceeds a threshold, in the descending order of the degree of association below the topic.

The determination function 157, among second medical data that includes one or more pieces of data representing the condition of a subject to be examined and that is included in the medical information, determines the display layout for displaying the data related to the one topic selected by the selection function 155.

Specifically, first, in cooperation with the second calculation function 156, the determination function 157 calculates the degree of association between the topic selected by the selection function 155 (selected topic) and the text data included in the sample test data, vitals data, prescription data, nursing record data, and the like, or the metadata indicating the attribute of the medical image data, using a known analysis method similar to that used for calculating the degree of association between the topics.

Then, the determination function 157 determines the medical data (for example, the second medical data) such as the sample test data, vitals data, medical image data, prescription data, and nursing record data including the text data (or metadata) in which the degree of association with the selected topic exceeds a threshold, as a display candidate to be displayed on the data display screen DD.

Next, the determination function 157 refers to the display information stored in the memory 120, and checks the screen size of the display device 140 that displays the data display screen DD. Moreover, the determination function 157 checks the classification information of each medical data determined as a display candidate. Then, the determination function 157 determines the display size and display position of the medical data, on the basis of the degree of association with the selected topic, the classification information of the medical data, and the screen size of the display device 140.

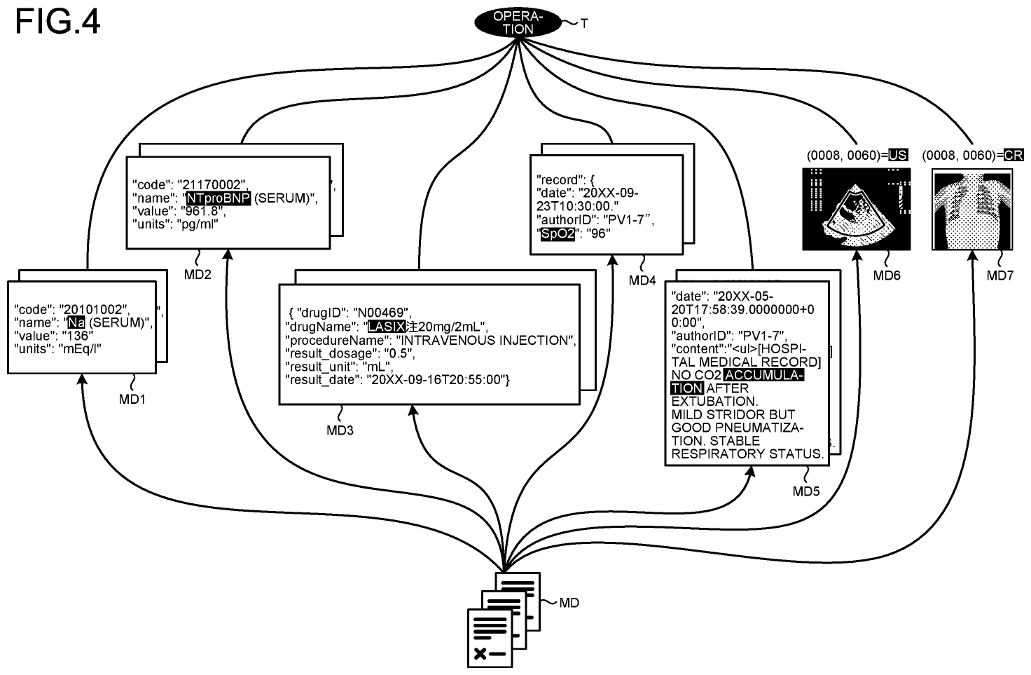

Hereinafter, a determination process of the display layout and a display process of the medical data will be described with reference to FIG. 4 to FIG. 7. FIG. 4 is an image illustrating an example of a calculation process of the degree of association between the topic and medical data.

In this example, a case when the user places the cursor CS on “topic: operation” in FIG. 3 and left clicks the mouse will be described. In this case, the selection function 155 selects the “topic: operation”. Next, the determination function 157 calculates the degree of association between the “topic: operation” and the medical data such as the sample test data of the patient to be examined identified by the “patient ID: P001”.

For example, in cooperation with the second calculation function 156, the determination function 157 calculates the degree of association between the “topic: operation” T and the character string “Na” included in “sample test data: Na” MD1. This numerical value is the degree of association between the “topic: operation” T and the “sample test data: Na” MD1. If the degree of association between the “topic: operation” T and the “sample test data: Na” MD1 exceeds a threshold, the determination function 157 determines the “sample test data: Na” MD1 as a display candidate.

The determination function 157 also performs the same process on “sample test data: NTproBNP” MD2, “prescription data: Lasix” MD3, “vitals data: SpO2” MD4, “nursing record data: 20XX-05-20” MD5, “medical image data: US” MD6, and “medical image data: CR” MD7.

Next, the determination function 157 refers to the classification information stored in the memory 120, and checks the classification information of each medical data having become a display candidate.

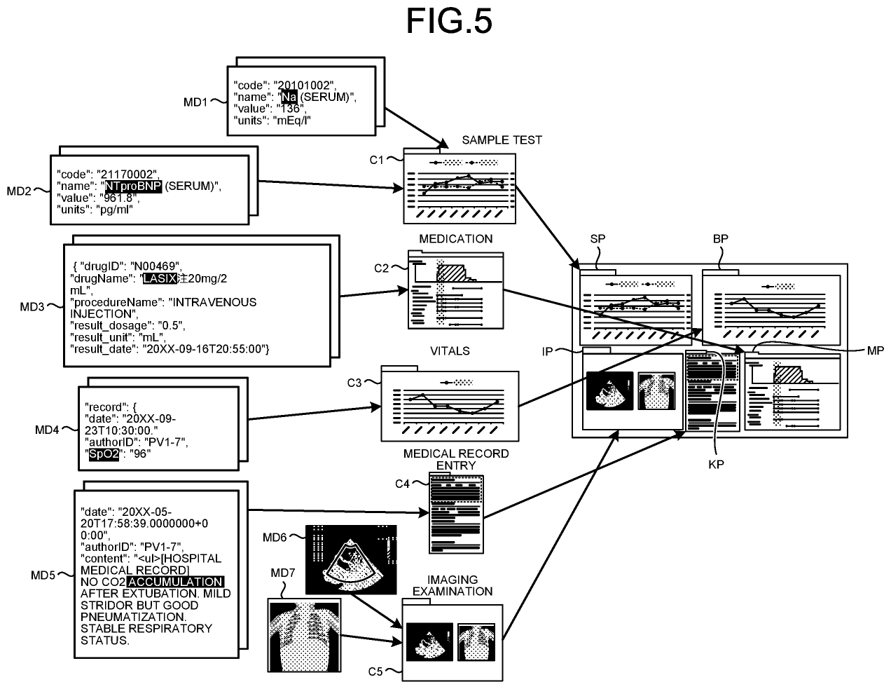

FIG. 5 is an image illustrating an example of medical data classification and medical data display. In this example, the medical data are the “sample test data: Na” MD1, “sample test data: NTproBNP” MD2, “prescription data: Lasix” MD3, “vitals data: SpO2” MD4, “nursing record data: 20XX-05-20” MD5, “medical image data: US” MD6, and “medical image data: CR” MD7.

For example, the determination function 157 refers to the classification information and confirms that the “sample test data: Na” MD1 and the “sample test data: NTproBNP” MD2 belong to the “classification: sample test” C1. Similarly, the determination function 157 confirms that the “prescription data: Lasix” MD3 belongs to the “classification: medication” C2.

Similarly, the determination function 157 confirms that the “vitals data: SpO2” MD4 belongs to the classification of “classification: vitals” C3. Similarly, the determination function 157 confirms that the “nursing record data: 20XX-05-20” MD5 belongs to the classification of “classification: medical record entry” C4. Similarly, the determination function 157 confirms that the “medical image data: US” MD6 and the “medical image data: CR” MD7 belong to the classification of “classification: imaging examination” C5.

Then, the determination function 157 determines the display arrangement of each medical data such that the medical data of “classification: sample test” C1 is displayed in the “sample test panel” SP, the medical data of “classification: medication” C2 is displayed in the “medication panel” MP, the medical data of “classification: vitals” C3 is displayed in the “vitals panel” BP, the medical data of “classification: medical record entry” C4 is displayed in the “medical record entry panel” KP, and the medical data of “classification: imaging examination” C5 is displayed in the “imaging examination panel” IP.

The display size of each panel, the arrangement of each panel, the display size of each medical data, and the arrangement of each medical data are determined by the determination function 157, on the basis of the degree of association between the topic and the medical data, the screen size of the display device 140, and the like. How the determination function 157 determines the layout of the data display screen will now be described in detail.

If the medical data as a display candidate is determined, the determination function 157 refers to the display information and the item number information stored in the memory 120, checks the maximum number of displayable medical data (items) on the display device 140 used for display, and determines the display item indicating the item to be displayed on the data display screen. If the number of medical data as display candidates is less than the number of displayable medical data on the display device 140, the determination function 157 determines all the medical data serving as display candidates as display items.

On the other hand, if the number of medical data as display candidates exceeds the number of displayable medical data, the determination function 157 arranges the medical data as display candidates in the descending order of the degree of association with the selected topic, and determines the medical data up to the “maximum number of displayable medical data” as the display items of the medical data.

In this manner, by setting the “maximum number of displayable medical data” for each screen size of the display device, situations can be prevented where the display size of each medical data becomes too small due to a large number of medical data related to the selected topic, and the user cannot easily understand the content of the medical data.

In the present embodiment, the maximum number of displayable medical data on the data display screen is determined according to the screen size. However, the number of medical data to be displayed on the data display screen may be set manually by the user.

When the display items are determined, the determination function 157 determines the arrangement and size of the data display panel (such as the “sample test panel”), on the basis of the number of medical data for each classification and the degree of association with the selected topic. For example, the determination function 157 increases the size of the data display panel, with an increase in the number of medical data for each classification.

Moreover, for example, the determination function 157 increases the size of the data display panel, with an increase in the degree of association of the medical data classified into the data display panel, with the selected topic. Furthermore, for example, the determination function 157 calculates the average value of the degree of association between the selected topic and the medical data for each classification, and arranges the data display panels from left in the descending order of the average value of the degree of association.

In this manner, by displaying the data display panel by changing the arrangement and display size of each medical data according to the degree of association between the selected topic and the medical data and the like, important information can be easily seen within a visual field, and the user can efficiently refer to the medical information.

When the arrangement of the data display panel or the like is determined, the determination function 157 determines the arrangement and the size of each medical data. For example, the determination function 157 increases the display size of medical data, with an increase in the degree of association of the medical data with the selected topic. Moreover, for example, the determination function 157 arranges the medical data such that the medical data having a higher degree of association with the selected topic is displayed on the upper part of the data display panel.

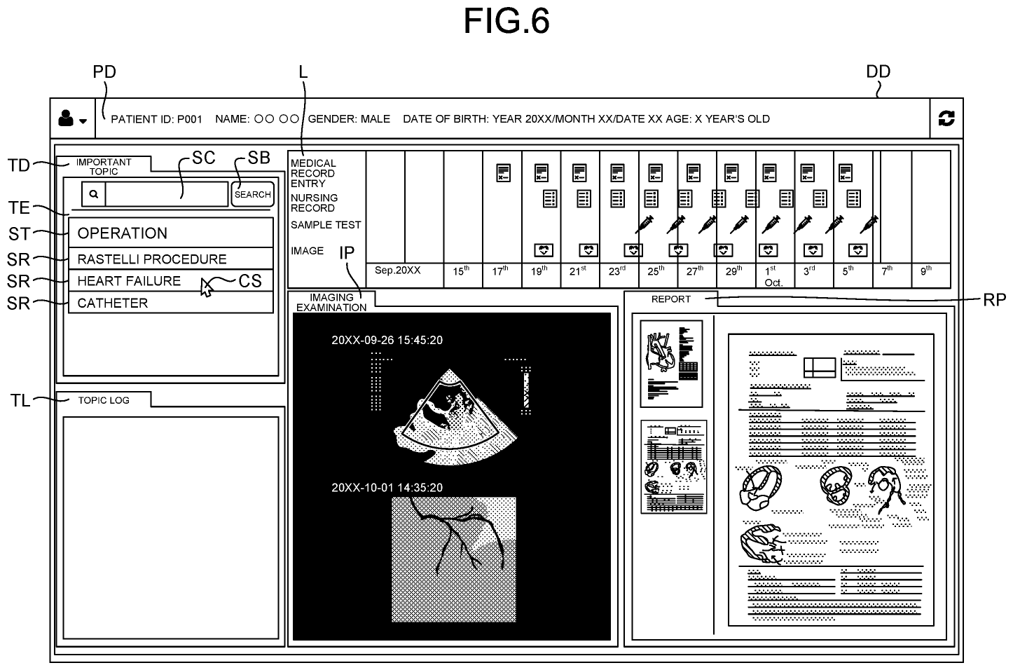

Hereinafter, a determination process of the display layout and a display process of the medical data will be described in detail with reference to FIG. 6 and FIG. 7. FIG. 6 and FIG. 7 are diagrams each illustrating an example of a data display screen.

For example, in the example of FIG. 6, if the selection function 155 selects “topic: operation”, the display control function 151 first displays the selected topic ST (in this example, “topic: operation”) on the upper most part of the topic display area TE.

Moreover, the display control function 151 displays the topic in which the degree of association with the “topic: operation” exceeds a threshold, below the selected topic ST, as the related topic SR (in this example, “topic: Rastelli procedure”, “topic: heart failure”, and “topic: catheter”) of the selected topic ST.

In this manner, if the topic to which the user wishes to refer is displayed as the related topic SR of the selected topic ST, the user does not have to collect the medical data, and can efficiently refer to the necessary medical information. Moreover, by automatically displaying the related topic SR of the selected topic ST, existence of other viewpoints can be informed to a user, and the probability of occurrence of overlooking important medical data can be reduced.

Next, in cooperation with the second calculation function 156, the determination function 157 calculates the degree of association between the “topic: operation” and each medical data, and determines the medical data in which the degree of association is equal to or more than a threshold as a display candidate. In this example, the determination function 157 determines two items of medical data in the “classification: imaging examination” and two items of medical data in the “classification: report” (four items in total) as display candidates.

Next, the determination function 157 refers to the display information and the item number information stored in the memory 120, and checks the screen size of the display device 140 and the maximum number of displayable medical data. For example, if the maximum number of displayable medical data is “10”, because the display candidates are four items, the determination function 157 determines all the display candidates as display items.

Next, the determination function 157 determines the arrangement and the arrangement size of the “imaging examination panel” IP and the “report panel” RP, on the basis of the degree of association between the “topic: operation” and each medical data, the number of medical data in the “classification: imaging examination”, and the number of medical data in the “classification: report”.

Moreover, the determination function 157 determines the arrangement and the display size of each medical image data in the “imaging examination panel” IP, on the basis of the degree of association between the “topic: operation” and each medical image data. Furthermore, the determination function 157 determines the arrangement and the display size of each report data in the “report panel” RP, on the basis of the degree of association between the “topic: operation” and each report data.

When the determination function 157 determines the layout of the data display screen, the display control function 151 performs control of displaying the data display screen DD on the display device 140, according to the layout determined by the determination function 157.

For example, as illustrated in FIG. 6, the display control function 151 displays the patient data field PD, the topic display panel TD, the topic log panel TL, the medical data list L, the “imaging examination panel” IP, and the “report panel” RP, on the data display screen DD displayed on the display device 140.

Because the patient data field PD, the topic display panel TD, and the medical data list L are as described above, the description thereof will be omitted. The “imaging examination panel” IP is a panel for displaying medical data classified into the “classification: imaging examination”.

The “report panel” RP is a panel for displaying the medical data classified into the “classification: report”. The left side of the panel represents the list of reports, and the user can select any one of the reports. The right side of the panel is an enlarged display field, and the display control function 151 enlarges and displays one of the reports, which is selected by the user from the reports at the right side.

If the user places the cursor CS on another topic displayed on the topic display panel TD and left-clicks the mouse, the selection function 155 selects the topic, and the determination function 157 determines the layout again. Hence, according to the screen layout determined again by the determination function 157, the display control function 151 displays the data display screen DD.

For example, if the selection function 155 selects the “topic: heart failure”, which is one of the topics T, as illustrated in FIG. 7, the display control function 151 displays the medical data related to the “topic: heart failure” on the data display screen DD, according to the screen layout determined by the determination function 157.

In the example of FIG. 7, according to the screen layout determined by the determination function 157, the display control function 151 displays the medical data belonging to the “classification: sample test” in the “sample test panel” SP, the medical data belonging to the “classification: imaging examination” in the “imaging examination panel” IP, the medical data belonging to the “classification: vitals” in the “vitals panel” BP, and the medical data belonging to the “classification: medical record entry” in the “medical record entry panel” KP.

Moreover, the display control function 151 not only performs control of switching the display of medical data to the one related to the selected topic, but also performs control of changing the display of the topic log panel TL.

For example, the display control function 151 displays the past topic (in the example of FIG. 7, “topic: operation”), which is referred to before the current selected topic ST is referred to, in the topic log panel TL. Then, the display control function 151 displays the selection button LB for selecting the past topic and the reduction display PV of the data display screen, which is displayed when the past topic is selected, on the topic log panel TL.

In this example, the display control function 151 displays only one past topic. However, if there are a plurality of topics that have been referred to in the past, the display control function 151 may also display a plurality of the past topics. In this manner, by displaying the past topics in a selectable manner, the user can efficiently refer to the medical information when the user wishes to go back to the original display to check the information.

Returning to FIG. 1, the description will continue. The search function 158 searches for a topic at least a part of which matches with the character string specified by the user, from the topics extracted by the extraction function 153. Moreover, the search function 158 searches for a topic equivalent or synonymous to the character string specified by the user, from the topics extracted by the extraction function 153.

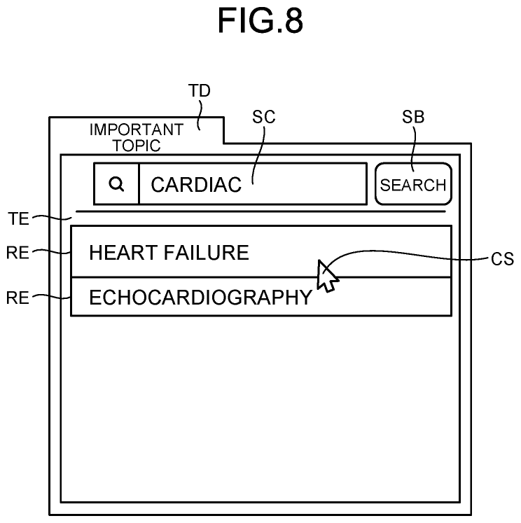

A topic search process will now be described with reference to FIG. 8. FIG. 8 is an image illustrating an example of a topic search process.

In this example, the user enters a word “cardiac” in the search box SC using a keyboard or the like, and presses the search button SB. In this case, the search function 158 searches for the topic including “cardiac”, from the topics extracted by the extraction function 153. Moreover, the search function 158 searches for the topic including “Heart”, which is a term equivalent to the “cardiac”.

The display control function 151 displays “topic: heart failure” and “topic: echocardiography”, which are topics RE hit in the search in the topic display area TE, in the descending order of the level of importance calculated by the first calculation function 154. The user can select the topic by placing the cursor CS on the topic RE hit in the search and left clicking the mouse.

In this manner, by providing the search function 158, the user can efficiently find the topic he/she wishes to refer to, when too many topics are extracted by the extraction function 153.

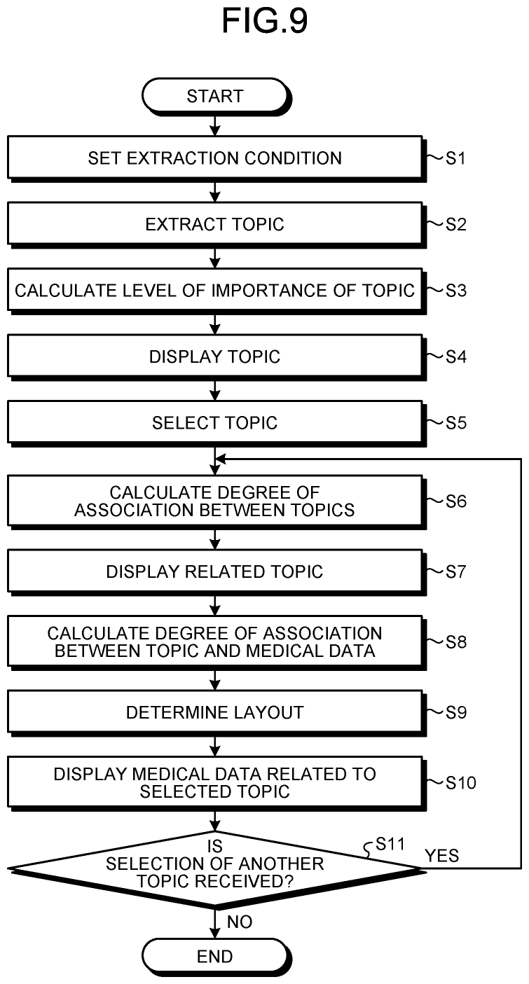

Next, a process executed by the medical information processing apparatus 100 according to the present embodiment will be described. FIG. 9 is a flowchart illustrating an example of a process executed by the medical information processing apparatus 100 according to the first embodiment.

First, the setting function 152 sets a topic extraction condition according to the user's instruction (step S1).

Next, according to the topic extraction condition set by the setting function 152, the extraction function 153 extracts a character string having clinical meaning from the text data included in the medical data, as a topic (step S2).

Next, the first calculation function 154 calculates the level of importance of the topic extracted by the extraction function 153 (step S3).

Next, the display control function 151 performs control of displaying the topics extracted by the extraction function 153 on the display device 140, in the descending order of the level of importance calculated by the first calculation function 154 (step S4).

Next, the selection function 155 selects one of the topics extracted by the extraction function 153 according to the user input (step S5).

Next, the second calculation function 156 calculates the degree of association between the topic selected by the selection function 155 (selected topic) and another topic (step S6).

Next, the display control function 151 performs control of displaying the topic in which the degree of association with the selected topic calculated by the second calculation function 156 exceeds a threshold, below the selected topic as the related topic (step S7).

Next, in cooperation with the second calculation function 156, the determination function 157 calculates the degree of association between the selected topic and the medical data on the patient to be examined (step S8).

Next, the determination function 157 refers to the display information stored in the memory 120, and checks the screen size of the display device 140. Then, the determination function 157 determines the layout of the data display screen (such as the arrangement and the display size of the medical data), on the basis of the screen size of the display device 140 and the degree of association with the selected topic (step S9).

Next, the display control function 151 displays the medical data related to the selected topic on the data display screen, according to the determination made by the determination function 157 (step S10).

Next, the selection function 155 checks whether a selection input of another topic different from the currently selected topic is received from the user (step S11). If the selection input of another topic is received (Yes at step S11), the selection function 155 selects the other topic selected by the user, and proceeds to the process at step S6. On the other hand, if the selection input of another topic is not received (No at step S11), the present process is terminated.

The medical information processing apparatus 100 according to the first embodiment described above includes the extraction function 153 that extracts a character string having clinical meaning from the first medical data indicating the text data included in the medical information, which indicates various types of data used for medical treatment, as a topic that indicates the reference viewpoint of the medical information; the selection function 155 that selects one topic from the topics extracted by the extraction function 153; and the display control function 151 that, among the second medical data that includes one or more pieces of data representing the condition of the subject to be examined and that is included in the medical information, performs control of displaying data related to the one topic selected by the selection function 155 on the display device.

In this manner, because the character string having clinical meaning is automatically extracted as a topic, the user can check the medical information related to the topic on the screen, by simply selecting the topic. If there is a topic that matches with the reference purpose of the medical information, the user does not have to collect medical information according to his/her reference purpose. Therefore, with the medical information processing apparatus 100 according to the present embodiment, the user can efficiently refer to the necessary medical information.

Moreover, if the user can efficiently refer to the necessary medical information, the user can smoothly make decisions on medical treatment such as making a diagnosis and determining the treatment policy.

### Second Embodiment

The second embodiment is based on the first embodiment. The second embodiment is different from the first embodiment in that the medical information processing apparatus 100 includes an aggregation function 159. Hereinafter, the difference between the second embodiment and the first embodiment will be mainly explained, and the repeated explanation will be omitted as appropriate.

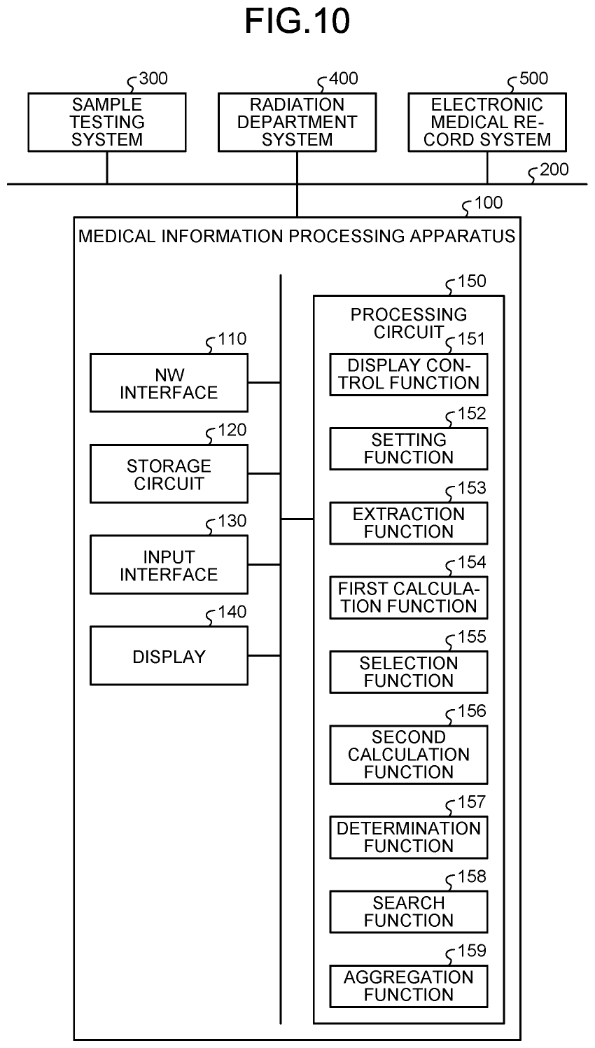

FIG. 10 is a block diagram illustrating an example of a configuration of the medical information processing apparatus 100 according to a second embodiment. The processing circuit 150 of the medical information processing apparatus 100 according to the present embodiment further includes the aggregation function 159.

The aggregation function 159 analyzes the character string having clinical meaning extracted by the extraction function 153, and aggregates the equivalent terms, synonyms, and abbreviations into one topic. Specifically, by using known medical ontologies, thesauruses, dictionaries, and the like, the aggregation function 159 aggregates words such as equivalent terms, synonyms, superordinate/subordinate concept words, and abbreviations-official names into one topic, on the basis of the clinical meaning of the topic.

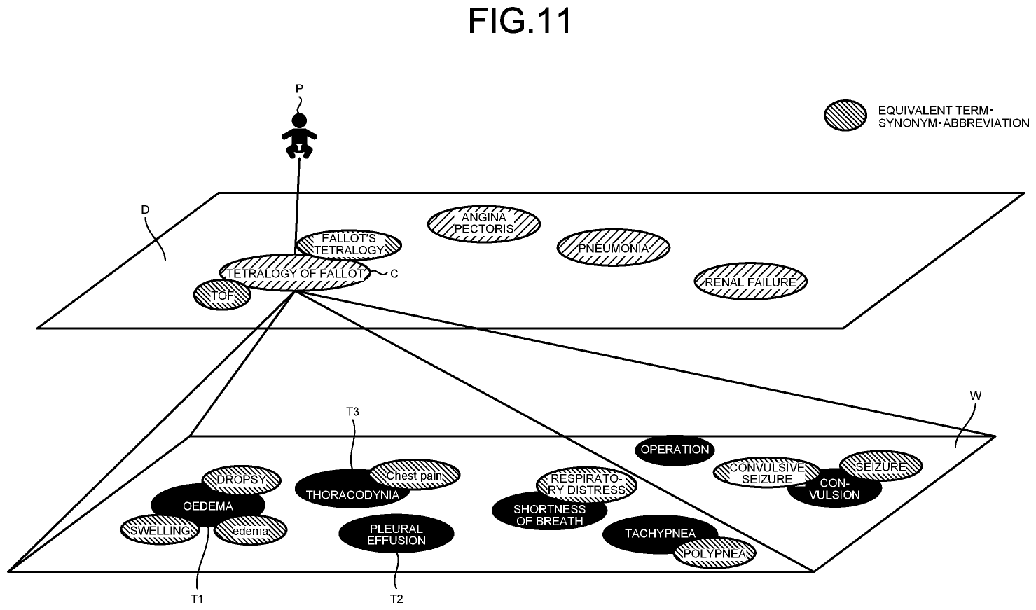

Hereinafter, an aggregation process of topics will be described with reference to FIG. 11. FIG. 11 is an image illustrating an example of an aggregation process of topics.

In this example, a process to be performed subsequent to the process of extracting the character string W having clinical meaning such as oedema, thoracodynia, and shortness of breath as a topic in FIG. 2 will be described. First, for example, by using known medical ontologies, thesauruses, dictionaries, and the like, the aggregation function 159 recognizes that “oedema”, “dropsy”, “swelling”, and “edema” are terms equivalent to each other.

Next, the aggregation function 159 checks the level of importance of “oedema”, “dropsy”, “swelling”, and “edema” calculated by the first calculation function 154. Then, for example, if “oedema” has the highest level of importance among “oedema”, “dropsy”, “swelling”, and “edema”, the aggregation function 159 aggregates “oedema”, “dropsy”, “swelling”, and “edema” into one “topic: oedema” T1, using “oedema” as a topic name.

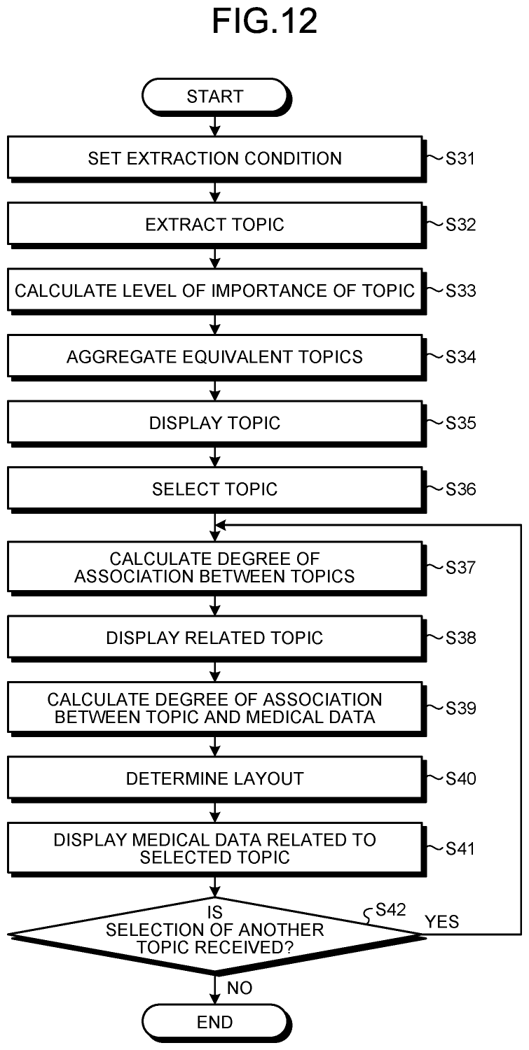

Next, a process executed by the medical information processing apparatus 100 according to the present embodiment will be described. FIG. 12 is a flowchart illustrating an example of a process executed by the medical information processing apparatus 100 according to the second embodiment.

First, because the processes from step S31 to step S33 are the same as those from step S1 to step S3 in FIG. 9, the description thereof will be omitted.

Subsequent to the process at step S33, the aggregation function 159 aggregates topics such as equivalent terms, synonyms, superordinate/subordinate concept words, and abbreviations-official names into one topic, on the basis of the clinical meaning of the topic extracted by the extraction function 153 (step S34). In this process, the aggregation function 159 uses the name of the topic having the highest level of importance among the topics aggregated into one, as a topic name.

Moreover, because the processes subsequent to step S35 are the same as those from step S4 to step S11 in FIG. 9, the description thereof will be omitted.

The medical information processing apparatus 100 according to the second embodiment described above includes the aggregation function 159 that analyzes the character string having clinical meaning extracted by the extraction function 153, and that aggregates the equivalent terms, synonyms, and abbreviations as one topic.

In this manner, because the topics such as equivalent terms and synonyms are aggregated into one topic, when a large number of equivalent and synonymous topics are in the topics extracted by the extraction function 153, the user can easily find the topic that the user wishes to find. Therefore, with the medical information processing apparatus 100 according to the present embodiment, the user can efficiently refer to the necessary medical information.

### Third Embodiment

The third embodiment is based on the second embodiment. The third embodiment is different from the second embodiment in that the medical information processing apparatus 100 includes a generation function 160. Hereinafter, the difference between the third embodiment and the second embodiment will be mainly explained, and the repeated explanation will be omitted as appropriate.

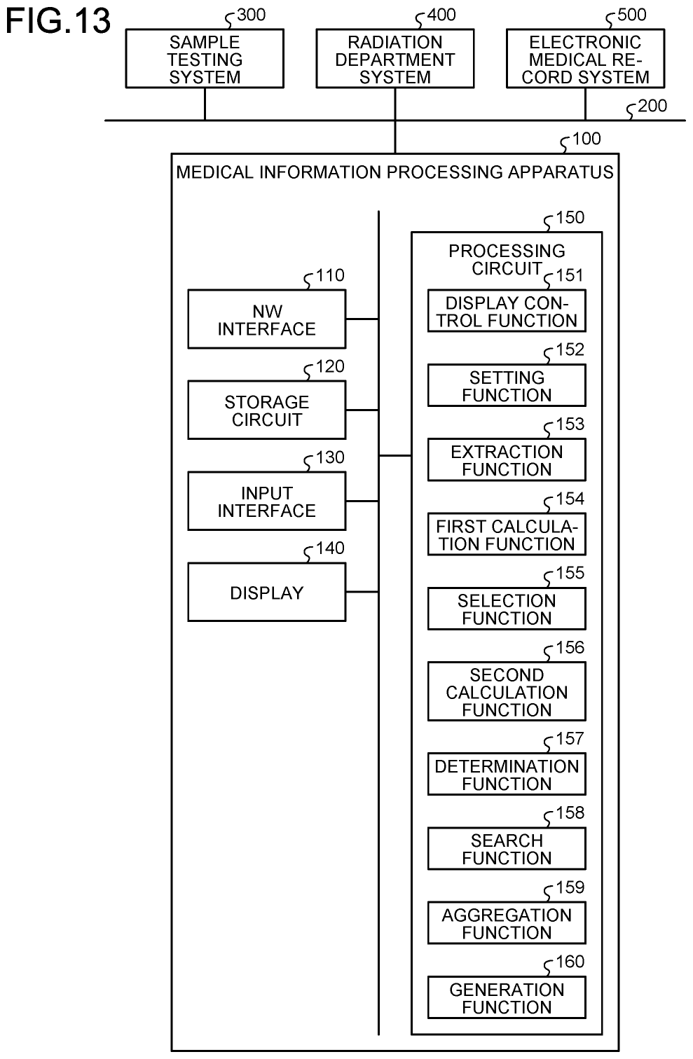

FIG. 13 is a block diagram illustrating an example of a configuration of the medical information processing apparatus 100 according to a third embodiment. The processing circuit 150 of the medical information processing apparatus 100 according to the present embodiment further includes the generation function 160.

In the present embodiment, the second calculation function 156 calculates the degree of association with each of the topics aggregated by the aggregation function 159. The second calculation function 156 may also calculate the degree of association between the topics before being aggregated.

The generation function 160 generates a topic group, by aggregating one or more topics according to the degree of association calculated by the second calculation function 156. Specifically, first, the generation function 160 aggregates the topics in which the degree of association calculated by the second calculation function 156 indicates equal to or more than a threshold. Next, the generation function 160 checks the level of importance calculated by the first calculation function 154 for each aggregated topic.

Then, the generation function 160 generates a topic group including the aggregated topics, using the name of the topic having the highest level of importance among the aggregated topics, as the name of the topic group. The topic group may only include one topic. Moreover, the generation function 160 may only generate a topic group, without aggregating the topics such as equivalent terms.

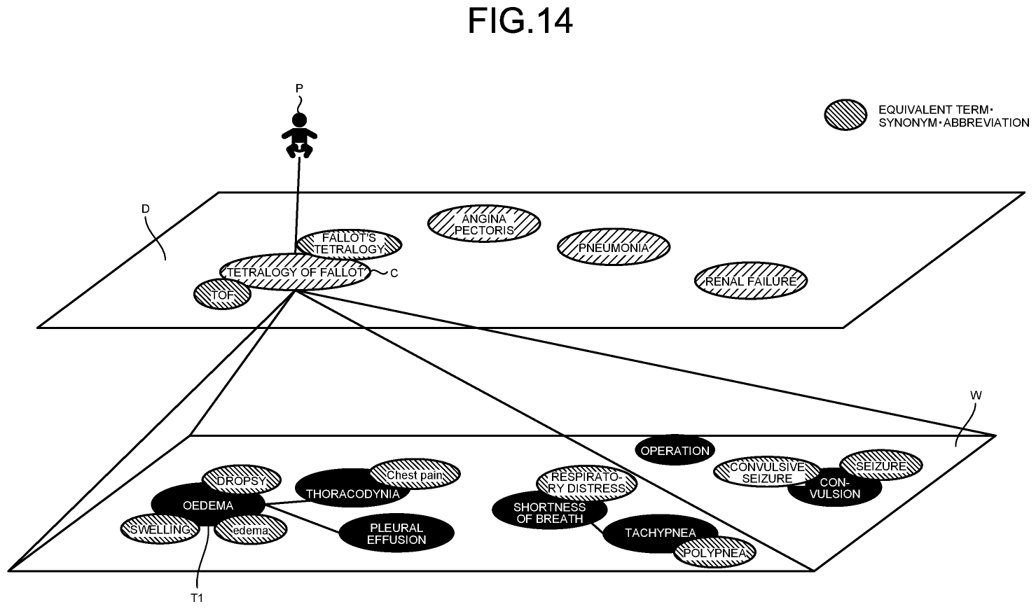

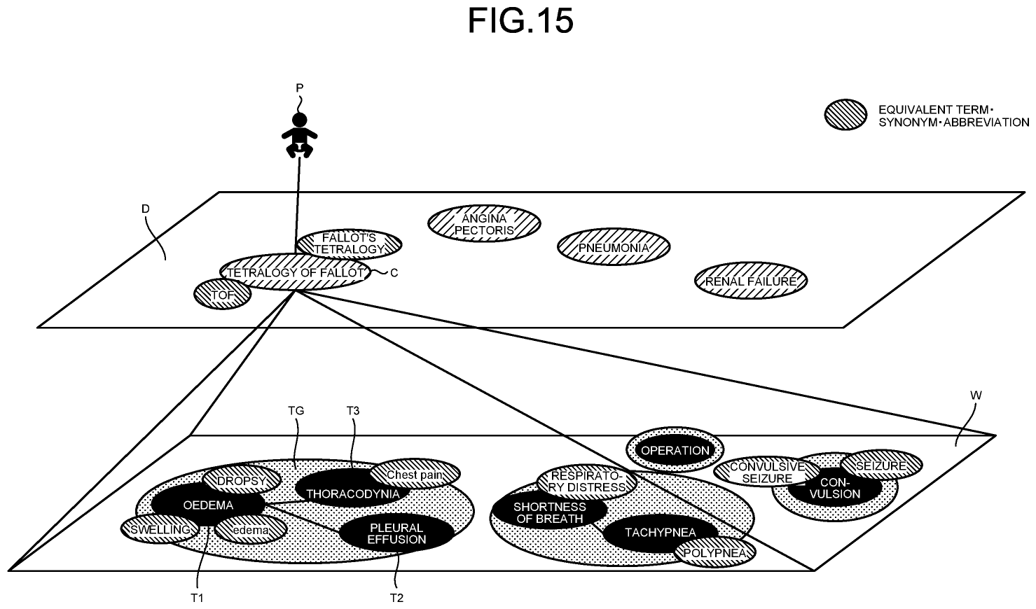

Hereinafter, a generation process of a topic group will be described with reference to FIG. 14 and FIG. 15. FIG. 14 is an image illustrating an example of an aggregation process of related topics. FIG. 15 is an image illustrating an example of a generation process of a topic group.

In this example, a process to be performed subsequent to the process of aggregating the topics such as equivalent terms in FIG. 11 will be described. First, for example, the second calculation function 156 calculates the degree of association between the topics such as “topic: oedema” T1—“topic: pleural effusion” T2, “topic: oedema” T1—“topic: thoracodynia” T3, and the like using a known analysis method such as association analysis.

For example, if the degree of association between the topics of “topic: oedema” T1—“topic: pleural effusion” T2 and “topic: oedema” T1—“topic: thoracodynia” T3 is equal to or more than a threshold, as illustrated in FIG. 14, the generation function 160 aggregates the “topic: oedema” T1, the “topic: pleural effusion” T2, and the “topic: thoracodynia” T3.

Next, the generation function 160 checks the level of importance of “oedema”, “pleural effusion”, and “thoracodynia” calculated by the first calculation function 154. Then, for example, if “oedema” has the highest level of importance among “oedema”, “pleural effusion”, and “thoracodynia”, as illustrated in FIG. 15, the generation function 160 generates “topic group: oedema” TG including the “topic: oedema” T1, the “topic: pleural effusion” T2, and the “topic: thoracodynia” T3, using “oedema” as a topic group name.

In this example, the “topic: oedema” T1, which is the topic group name, is referred to as a heading topic, and the “topic: pleural effusion” T2 and the “topic: thoracodynia” T3 are referred to as related topics of the heading topic.

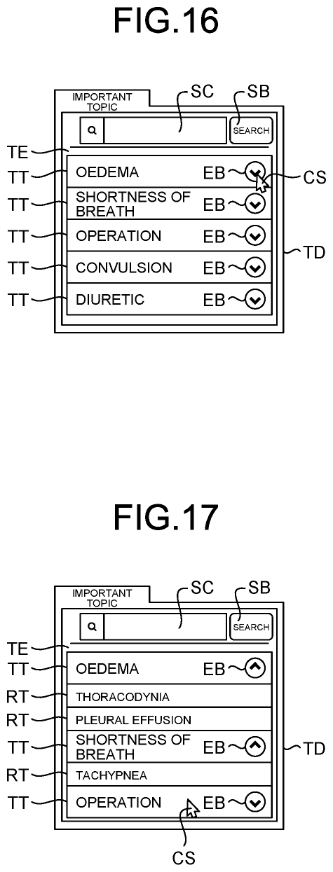

When the generation function 160 generates a topic group, the display control function 151 performs control of displaying topics on the display device 140. Hereinafter, a display process of topics will be described with reference to FIG. 16 and FIG. 17. FIG. 16 and FIG. 17 are images each illustrating an example of a topic display screen.

When the generation function 160 generates a topic group, the display control function 151 displays the topic display panel TD as illustrated in FIG. 16 on the display device 140.

In this example, the display control function 151 displays “oedema”, “shortness of breath”, “operation”, “convulsion”, and “diuretic” as the heading topics TT in the topic display area TE, in the descending order of the level of importance calculated by the first calculation function 154. Moreover, the display control function 151 displays the expansion button EB at the right end of each of the heading topics TT.

For example, if the user places the cursor CS on the expansion button EB and left-clicks the mouse, as illustrated in FIG. 17, the display control function 151 displays the related topic RT of the heading topic in the topic display area TE. In the example of FIG. 17, the display control function 151 displays “thoracodynia” and “pleural effusion” as the related topics RT of the “topic: oedema”, which is the heading topic TT, in the descending order of the level of importance calculated by the first calculation function 154.

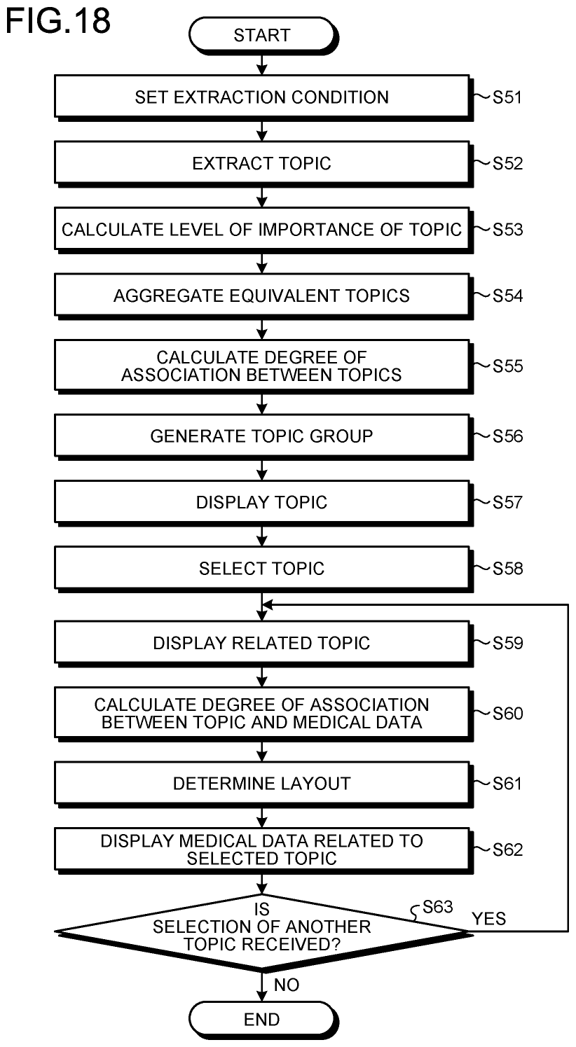

Next, a process executed by the medical information processing apparatus 100 according to the present embodiment will be described. FIG. 18 is a flowchart illustrating an example of a process executed by the medical information processing apparatus 100 according to the third embodiment.

First, because the processes from step S51 to step S54 are the same as those from step S31 to step S34 in FIG. 12, the description thereof will be omitted.

Subsequent to the process at step S54, the second calculation function 156 calculates the degree of association between the topics, for the topics extracted by the extraction function 153 and aggregated by the aggregation function 159 (step S55).

Next, the generation function 160 generates one or more topic groups, by aggregating the topics in which the degree of association calculated by the second calculation function 156 is equal to or more than a threshold (step S56). In this process, the generation function 160 uses the name of the topic having the highest level of importance in the generated topic group, as a topic group name.

Next, the display control function 151 performs control of displaying one or more topic group names generated by the generation function 160 on the display device 140 (step S57). In this process, the display control function 151 also performs control of displaying one or more topic names included in the topic group on the display device 140.

Next, the selection function 155 selects one of the topics extracted by the extraction function 153 according to the user's instruction (step S58).

Next, the display control function 151 performs control of displaying the topic in which the degree of association with the selected topic calculated by the second calculation function 156 exceeds a threshold, below the selected topic as the related topic (step S59).

Because the processes from step S60 to step S63 are the same as those from step S39 to step S42 in FIG. 12, the description thereof will be omitted.

The medical information processing apparatus 100 according to the third embodiment described above includes the generation function 160 that generates a topic group by aggregating one or more topics according to the degree of association calculated by the second calculation function 156.

In this manner, because the topic group in which the related topics are aggregated is generated, the user can easily find the related topic and efficiently refer to the necessary medical information.

The first embodiment to the third embodiment described above can also be modified and implemented as appropriate, by changing a part of the configuration or function of each device. Thus, in the following, some modifications according to the embodiments described above will be explained as other embodiments. In the following, points different from those in the embodiments described above will be mainly explained, and detailed description of points common to the above-described contents will be omitted. Moreover, the modifications described below can be implemented individually or in combination as appropriate.

First Modification

In the first embodiment to the third embodiment described above, by using a known analysis method, the determination function 157 calculates the degree of association between the selected topic and the medical data (item), and determines the medical data (item) in which the degree of association is equal to or more than a threshold as a display candidate to be displayed on the data display screen. In contrast, the determination function 157 may also determine the display candidate on the basis of correspondence information in which the topic is associated with one or more pieces of medical data (items).

The correspondence information described above is defined on the basis of clinical practice guidelines and the like. According to the present modification, the process of calculating the degree of association between the selected topic and the medical data (item) is not necessary. Hence, the load on the medical information processing apparatus 100 can be reduced.

Second Modification

In the first embodiment to the third embodiment described above, the determination function 157 determines the layout of the display screen. In contrast, the user may replace the medical data (item) or change the display size of the medical data (item), after the determination function 157 has determined the layout of the display screen. Moreover, any name can be applied to the layout modified by the user, and the modified layout can be stored in the memory 120 as a custom topic.

According to the present modification, the medical data (item) corresponding to the user's own viewpoint can be efficiently displayed.

According to at least one of the embodiments described above, the necessary medical information can be efficiently referred to.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus, comprising: a processing circuitry configured to
extract a character string having clinical meaning from first medical data indicating text data included in medical information, as a topic indicating a reference viewpoint of the medical information,
select one of the topics from the extracted topics according to a user input, and
among second medical data that includes one or more pieces of data representing a condition of a subject to be examined and that is included in the medical information, performs control of displaying the second medical data related to one of the selected topics on a display device.

2. The medical information processing apparatus according to claim 1, wherein the processing circuitry extracts the character string from the first medical data as the topic, according to an extraction condition indicating a condition for extracting the character string, corresponding to a reference purpose of the medical information.

3. The medical information processing apparatus according to claim 2, wherein the processing circuitry extracts the character string from the first medical data as the topic, according to the extraction condition including at least one of a disease name, a clinical department name, subject identification information for identifying a subject, and doctor identification information for identifying a doctor.

4. The medical information processing apparatus according to claim 1, wherein the processing circuitry calculates a level of importance indicating a degree of importance of the topic.

5. The information processing apparatus according to claim 4, wherein the processing circuitry performs control of rearranging and displaying the topics, according to the calculated level of importance of the topics.

6. The medical information processing apparatus according to claim 1, wherein the processing circuitry analyzes the extracted character string, and aggregates an equivalent term, synonym, and abbreviation as one topic.

7. The medical information processing apparatus according to claim 6, wherein the processing circuitry
calculates a degree of association indicating a degree of relation between the topics, and
generates a topic group by aggregating one or more topics according to the calculated degree of association.

8. The medical information processing apparatus according to claim 1, wherein the processing circuitry performs control of arranging the second medical data and displaying the second medical data on the display device, according to classification information that indicates a type of the second medical data.

9. The medical information processing apparatus according to claim 1, wherein the processing circuitry performs control of changing a display size and arrangement of the second medical data, according to size of the display device.

10. The medical information processing apparatus according to claim 1, wherein the processing circuitry
performs control of displaying a topic history indicating a topic that has been referred to before a topic currently referred to by a user, and
selects one of the topics from the topic history.

11. The medical information processing apparatus according to claim 1, wherein the processing circuitry searches for a topic at least a part of which matches with a character string specified by a user, from the extracted topics.

12. The medical information processing apparatus according to claim 11, wherein the processing circuitry searches for a topic equivalent or synonymous to the character string specified by the user.

13. The medical information processing apparatus according to claim 1, wherein the processing circuitry performs control of displaying one or more pieces of data corresponding to one of the selected topics on a display device, based on correspondence information in which the topic is associated with one or more pieces of the second medical data.
